(19) United States
(12) Patent Application Publication
(10) Pub. No.: US 2025/0228618 A1
(43) Pub. Date: Jul. 17, 2025

(54) METHODS FOR PLANNING ENDOVASCULAR PROCEDURES ACCESSING THE INTRACRANIAL SUBARACHNOID SPACE

(71) Applicant: CEREVASC, INC., Charlestown, MA (US)

(72) Inventor: Adel M. Malek, Weston, MA (US)

(73) Assignee: CEREVASC, INC., Charlestown, MA (US)

(21) Appl. No.: 19/015,502

(22) Filed: Jan. 9, 2025

Related U.S. Application Data
(60) Provisional application No. 63/620,397, filed on Jan. 12, 2024.

(51) Int. Cl. A61B 34/10; A61M 27/00

(52) U.S. Cl. CPC A61B 34/10; A61B 2034/104; A61B 2034/105; A61B 2034/107; A61M 27/006

(57) ABSTRACT

Methods for planning endovascular procedures intended to access extravascular spaces are disclosed herein for measuring anatomical distances and assessing such distance relative to anatomical screening criteria specified for a procedure to improve patient safety and procedural success, for example, when planning a procedure for navigating a delivery catheter through a blood vessel wall to a location within the intracranial subarachnoid space.

### RELATED APPLICATION DATA

The present application claims the benefit or priority under 35 U.S.C. § 119 to Provisional Application Ser. No. 63/620,397, filed on Jan. 12, 2024.

### FIELD OF THE INVENTION

The present disclosure relates to procedure planning methods to facilitate endovascular procedures intended to access an extra-vascular location in the body. More particularly, the present disclosure pertains to procedure planning methods that assess patient imaging studies to approve or not approve patient anatomy for endovascular procedures to penetrate a blood vessel wall and access an extravascular space.

### BACKGROUND

There has been significant progress made in recent decades in the development and practice of minimally invasive surgical devices and procedures. As used herein, the term “minimally invasive” refers to the use of surgical devices and implants that access the vasculature of the body via arterial or venous access in the groin, arm or neck area, as opposed to more invasive traditional procedures that access the body more directly through percutaneous/solid tissue incisions and cutting and/or boring through bones, as needed to access the internal area of the body on which the procedure is performed. The term “minimally invasive” may also refer to the to the use of surgical devices and implants that access the body via other natural body orifices, cavities and tubular structures, such as the esophagus, intestines, bronchial passageways, etc. Percutaneous access through the skin into solid tissue, e.g., for accessing the liver, prostate or lungs, e.g., using a trocar and stylet, may also be considered minimally invasive.

One minimally invasive surgical technique, referred to as an endovascular CSF shunt delivery procedure, involves treating hydrocephalus, pseudotumor cerebri, and/or intracranial hypertension, which is caused by excess cerebrospinal fluid (CSF) fluid in the intracranial sub-arachnoid space (ISAS) of the patient—a naturally occurring separation between the pia mater and the arachnoid layer in the cranium of the patient where CSF pools. Typically, such CSF is passed into the ISAS over the cerebral hemispheres and then into the venous system by arachnoid granulations, where the CSF is reabsorbed into the body of the patient. The endovascular CSF shunt delivery procedure involves endovascularly creating an anastomosis between the lumen of the inferior petrosal sinus (IPS) (or other dural venous sinus) and the ISAS of the patient, implanting an endovascular shunt device within the anastomosis, and draining the excess CSF from the ISAS into the venous system of the patient via the implanted shunt device. Examples of endovascular devices and procedures to drain CSF from the CSF-filled SAS into the venous system of a patient are described in U.S. Pat. Nos. 10,272,230 and 10,765,846 and U.S. Patent Publication Nos. 2020/0030588 and 2024/0390643, the entire disclosures of which are expressly incorporated herein by reference as though set forth in full.

However, because the anatomies of patients relevant to endovascular CSF shunt delivery procedures differ, pre-procedure planning and intra-procedural decision support for each patient must be performed to ensure success of the endovascular CSF shunt delivery procedure while maximizing patient safety. There, thus, is a need to formulate an efficient and effective pre-procedure plan to screen a patient prior to performing an endovascular CSF shunt delivery procedure on such patient and/or support intra-procedural decision-making.

### SUMMARY

In accordance with an aspect of the present inventions, a method for planning an endovascular procedure to access an intracranial subarachnoid space (ISAS) (e.g., an cerebellopontine (CP) angle cistern) through a blood vessel wall (e.g., an intracranial venous sinus, such as an inferior petrosal sinus (IPS)) of a patient is provided.

The method comprises obtaining magnetic resonance (MR) imaging of the blood vessel and the ISAS, measuring in the MRI imaging an unobstructed depth of the ISAS at a target penetration site or a distance from a target penetration site of the blood vessel to one or more critical structures (e.g., an artery, a nerve, or a brainstem of the patient) within the ISAS, and approving or disapproving the patient for the endovascular procedure based on the measured unobstructed depth of the ISAS at the target penetration site or the measured distance from the target pentration site of the blood vessel to the one or more critical structures within the ISAS.

One method may further comprise measuring in the MRI imaging a diameter of the blood vessel the target penetration site, and approving or disapproving the patient for the endovascular procedure based on measured diameter of the blood vessel. For example, the measured blood vessel diameter may be compared to a largest outer diameter of a delivery catheter intended to be used in the endovascular procedure, and the patient approved or disapproved for the endovascular procedure based on the comparison.

Another method may further comprise obtaining computed tomography (CT) imaging of the blood vessel and the ISAS, assessing the CT imaging for the presence or absence of bony anatomy near the target penetration site of the blood vessel, and approving or disapproving the patient for the endovascular procedure based on assessment.

Still another method may further comprise obtaining a venogram of the blood vessel including the target penetration site, evaluating the venogram for a curvature of the blood vessel proximate the target penetration site, and approving or disapproving the patient for the endovascular procedure based on the evaluation. For example, the venogram may comprise acquiring a cone-beam computed tomography (CT) imaging of the blood vessel and ISAS, in which case, the method may further comprise evaluating the cone-beam CT imaging for a pathway of the blood vessel to determine whether a delivery catheter, when advanced through the pathway, will travel off-axis from the pathway at the target penetration site to access the ISAS, and approving the patient for the endovascular procedure based on whether the delivery catheter will travel off-axis from the pathway at the target penetration site to access the ISAS.

If the patient is approved for the endovascular procedure, the method may further comprise performing the endovascular procedure (e.g., deploying an endovascular cerebrospinal fluid (CSF) shunt within the ISAS and/or administering a therapeutic agent into the ISAS).

In accordance with another aspect of the present inventions, a method for planning an endovascular procedure to access an ISAS (e.g., a CP angle cistern) through a blood vessel wall (e.g., an intracranial venous sinus, such as an IPS) of a patient is provided.

The method comprises obtaining imaging of the blood vessel and the ISAS, and performing at least two of the diagnostic steps to obtain anatomic screening criteria: measuring in the imaging an unobstructed depth of the ISAS at the target penetration site; measuring in the imaging a distance from a target penetration site of the blood vessel to one or more critical structures within the ISAS; measuring in the imaging a diameter of the blood vessel the target penetration site; assessing the imaging for the presence or absence of bony anatomy near the target penetration site of the blood vessel, and evaluating the imaging for a curvature of the blood vessel proximate the target penetration site. The method further comprises approving or disapproving the patient for the endovascular procedure based on the anatomic screening criteria. In one method, at least three of the steps are performed to obtain the anatomic screening criteria.

If the patient is approved for the endovascular procedure, the method may further comprise performing the endovascular procedure (e.g., deploying an endovascular cerebrospinal fluid (CSF) shunt within the ISAS and/or administering a therapeutic agent into the ISAS).

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions need not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is an anterior view of a head of a human patient, particularly showing relevant vascular structures of the head;

FIG. 2 is a median sagittal view of a head of the human patient, particularly showing relevant vascular structures of the head and the intracranial subarachnoid space (ISAS);

FIG. 3 is another median sagittal view of a head of the human patient, particularly showing relevant vascular structures of the head and the ISAS;

FIG. 4 is an anterior view of a head of the human patient, particularly showing relevant vascular structures of the head with respect to the ISAS;

FIG. 5 is a plan view showing the geometry of a delivery catheter used in the endovascular CSF shunt deployment procedure.

FIG. 6 is a flow diagram illustrating one procedure planning method that evaluates patient anatomy relevant to an endovascular CSF shunt deployment procedure in accordance with the present inventions;

FIG. 7 is a magnetic resonant (MR) image of the relevant patient anatomy obtained in accordance with the procedure planning method of FIG. 6, particulary showing unsuitable patient anatomy for the endovascular CSF shunt deployment procedure;

FIG. 8 is are MR images of the relevant patient anatomy obtained in accordance with the procedure planning method of FIG. 6, particulary showing suitable patient anatomy for the endovascular CSF shunt deployment procedure;

FIG. 9A is a computed tomography (CT) image of the relevant patient anatomy obtained in accordance with the procedure planning method of FIG. 6, particulary showing suitable patient anatomy for the endovascular CSF shunt deployment procedure;

FIG. 9B is CT image of the relevant patient anatomy obtained in accordance with the procedure planning method of FIG. 6, particulary showing unsuitable patient anatomy for the endovascular CSF shunt deployment procedure;

FIG. 10A is a venogram of the relevant patient anatomy obtained in accordance with the procedure planning method of FIG. 6, particulary showing suitable patient anatomy for the endovascular CSF shunt deployment procedure;

FIG. 10B is a cone-beam CT image of the relevant patient anatomy obtained in accordance with the procedure planning method of FIG. 6, particulary showing suitable patient anatomy for the endovascular CSF shunt deployment procedure; and

FIG. 11 is three-dimensional (3D) reconstruction of the relevant patient obtained in accordance with the flow diagram of FIG. 3, particulary showing a suitable pathway and an unsuitable pathway for the endovascular CSF shunt deployment procedure.

### DETAILED DESCRIPTION OF THE ILLUSTRATED EMBODIMENTS

The disclosed procedure planning methods are described in connection with example endovascular procedures for navigating to and penetrating the wall of a venous vessel, e.g., an inferior petrosal sinus (IPS), to access a specific location within the intracranial subarachnoid space (ISAS), e.g., a cerebellopontine angle cistern (CP angle cistern). Additional details on such example endovascular procedures are disclosed in PCT Publication Nos. WO2016/070147, WO2017/075544, WO2018/071600, WO2019/173784, and WO2023/178077, which are expressly incorporated herein by reference. It should be appreciated, however, the procedure planning methods described disclosed herein can be used for any procedure conducted from within a venous or arterial lumen to access a location within the ISAS (e.g., penetrating from within the cavernous sinus, superior petrosal sinus, sagittal sinus, transverse sinus, sigmoid sinus, basilar artery, anterior inferior cerebrellar artery, or other arterial vessel to access an ISAS).

Imaging systems and methods are employed to provide valuable information to operators and clinicians for planning interventional and minimally invasive procedures conducted in the vasculature or surrounding tissues and spaces in the body. Imaging modalities, including magnetic resonance (MR) imaging, computed tomography (CT) imaging, angiography, rotational angiography, and three-dimensional (3D) roadmapping, facilitate visualization of features of interest within the body. The data obtained from various different imaging modalities are used to plan for and visualize the endovascular procedures prior to performing the endovascular procedures, and optionally as the endovascular procedures are conducted real-time, all in relation to anatomic landmarks near the specific intended procedure location.

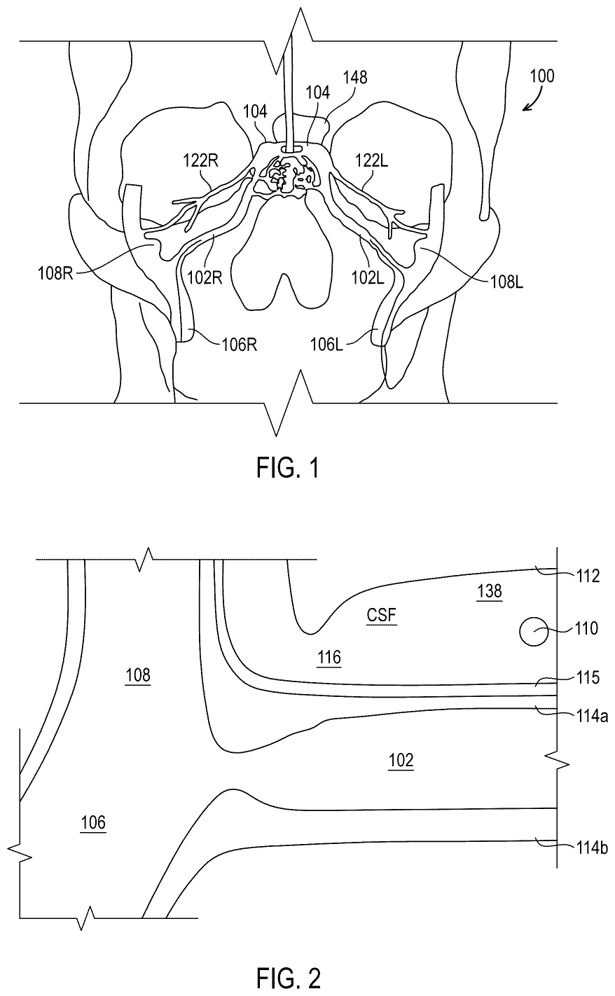

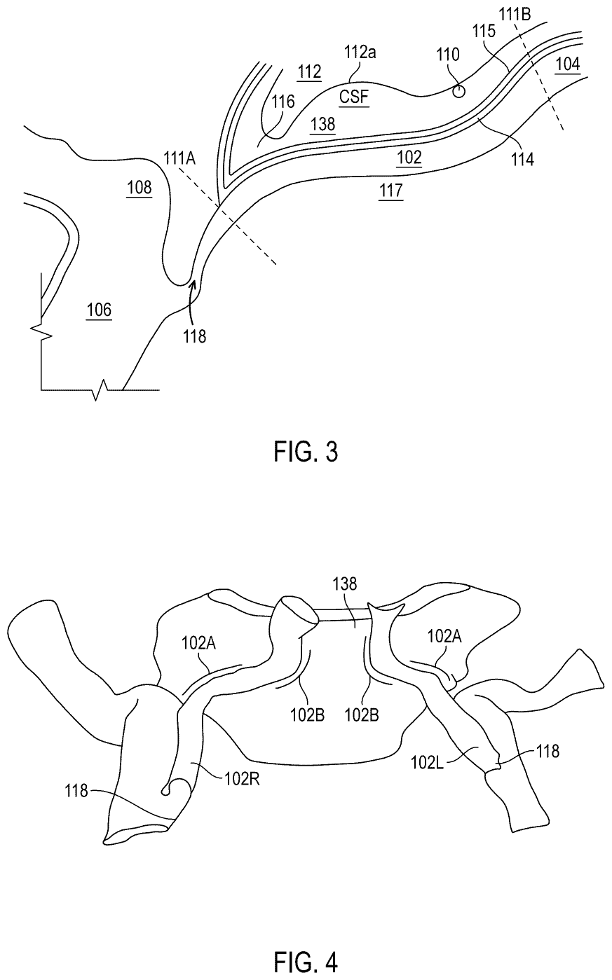

Referring first to FIGS. 1-2, within each side (i.e., the left side and the right side) of the patient's head 100, an inferior petrosal sinus (IPS) 102 (102L and 102R) connects a cavernous sinus (CS) 104 (104L and 104R) to a jugular vein (JV) 106 (106L and 106R) and/or a jugular bulb 108 (108L and 108R). The IPS 102 is a relatively small diameter intracranial venous sinus that facilitates drainage of cerebral venous blood into the JVs 106. In some patients, the junction of the IPS 102 and the JV 106 occurs within the jugular bulb 108. However, in other patients, this junction can occur at other locations in the JV 106. Moreover, while the IPS 102 is illustrated in FIGS. 1-2 as a single sinus passageway, in some patients, the IPS 102 can be a plexus of separate channels that connect the CS 104 to the JV 106 (not shown) and/or the jugular bulb 108. Within each side of the patient's head 100, a superior petrosal sinus (SPS) 122 (122L and 122R) also connects the CS 104 to a sigmoid sinus (distally located to jugular bulb 108).

The wall 114 of each IPS 102 is formed by a cylindrical layer of dura mater, which creates a hollow lumen through which blood flows. The IPS wall 114 may be topological divided between an upper IPS wall 114a outside of which an intracranial subarachnoid space (ISAS) 116, and in particular, a cerebellopointine angle cistern (CP angle cistern) 138, resides, and a lower IPS wall 114b formed by dura mater that sits in a channel in the clivus and/or petrous bone (not shown in FIGS. 1-2). The CP angle cistern 138 serves as a reservoir for cerebrospinal fluid (CSF) and, for the purposes of facilitating interventional techniques, may serve as a location for (i) deploying a portion of an endovascular cerebrospinal fluid shunt device, (ii) delivering therapeutic agents, or (iii) accessing and conducting other minimally invasive procedures. A cross-section of the IPS 102 orthogonal to the plane depicted in FIG. 2 would show that the cylindrical layer of dura mater forming a portion of the IPS 102 (i.e., the lower IPS wall 114b) is surrounded by clivus and/or petrous bone with the remaining portion of the circumference of the IPS 102 (i.e., the upper IPS wall 114a) covered by a delicate and avascular layer of arachnoid matter 115 (also referred to herein as the arachnoid layer) that faces the CP angle cistern 138. Arachnoid mater 115 is typically about 0.05 mm to 0.15 mm thick and is separated from the pia mater surrounding the brainstem 112 by the CSF-filled ISAS 116 (e.g., CP angle cistern 138).

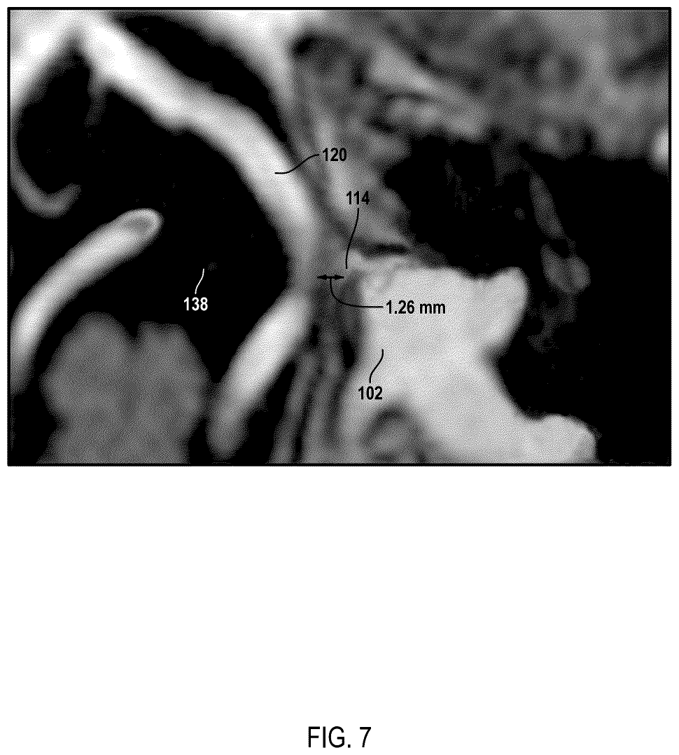

As illustrated in FIG. 5, embodiments of the disclosed procedure planning methods are described with respect to an endovascular CSF shunt deployment procedure that navigates a delivery catheter 10 including a penetrating element 250 through the venous vasculature to a target penetration site 12 in the IPS 102, penetrating through the upper IPS wall 114a and arachnoid layer 115 with the penetrating element of the delivery catheter 10 to access the CSF-filled CP angle cistern 138, and deploying the endovascular shunt 14 from the delivery catheter 10. The deployed shunt 14 facilitates CSF drainage from the CP angle cistern 138 into the venous system. In alternative embodiments, the CP angle cistern 138 may be accessed from the SPS 122 (FIG. 1).

When conducting such endovascular CSF shunt deployment procedures, it is paramount to avoid contacting or injurying critical structures within the ISAS 116 including, but not limited to, the brain stem, cranial nerves, basilar artery 110, and any other arterial structure or branch within the ISAS 116 and proximate the location where the delivery catheter penetrates the IPS wall 114 to access the CP angle cistern 138 (e.g., the vertebral artery, anterior inferior cerebellar artery (AICA), and posterior inferior cerebellar artery (PICA)). Embodiments of the procedure planning methods disclosed herein provide for screening of patient anatomy relevant to the procedure to assess whether a delivery catheter can safely access the CP angle cistern 138 without injuring any of the aforementioned critical structures within the ISAS 116.

For some patients, the clivus and/or petrous bone include portions or bony protuberances that extend more than 270° around the circumference of the IPS 102. And in certain patients, these bony portions or protuberances can frustrate or prevent a delivery catheter from accessing the CP angle cistern 138 by physically obstructing the delivery catheter from passing through IPS wall 114. This anatomical feature is an important procedure planning assessment to complete before or during the endovascular CSF shunt deployment procedure to ensure procedural success.

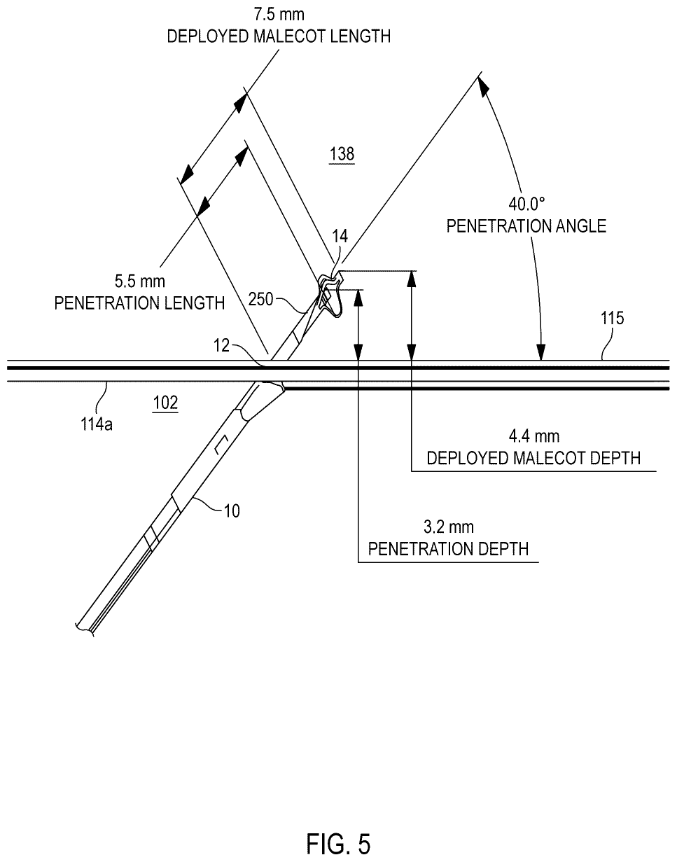

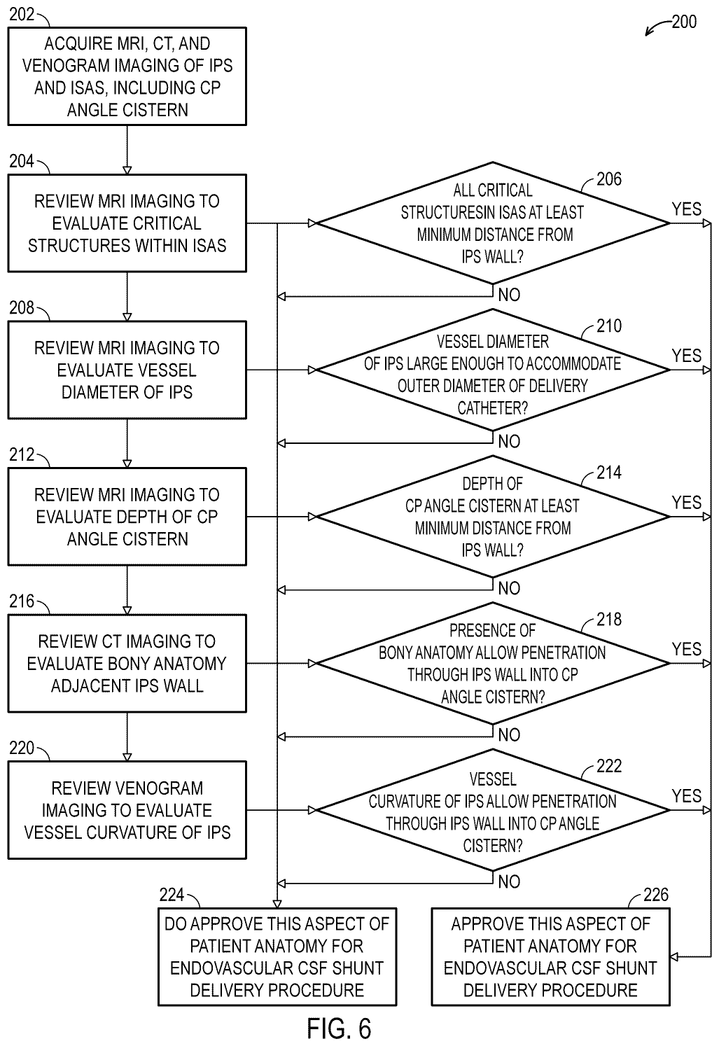

In many patients, the IPS 102 is coupled to the JV 106 at a location disposed below the jugular bulb 108, depicted as junction 118 in FIG. 3. The IPS 102 extends distally from the junction 118 in the medial wall of the JV 106, past the 9th cranial nerve 111A and jugular tubercle (not shown), while curving rostral-medially through a first curved portion 102A shown in FIG. 4, and then further curving medial-superiorly through a second curved portion 102B shown in FIG. 4 before connecting at the connection point 111B with the CS 104. The IPS 102 extends distally from the junction 118 through a curvature of approximately 25° to 100° or more in the first and second curved portions 102A and 102B until the IPS 102 connects with the CS 104. The CSF-filled CP angle cistern 138 lies immediately above the curved portion of the IPS 102.

The extent of vessel curvature of the IPS 102 through a first curved portion 102A shown in FIG. 4 is another important anatomical feature to assess when planning an endovascular CSF shunt shunt deployment procedure. Vessel curvature sufficient to allow the delivery catheter 10 to travel off-axis from the vessel path of the IPS 102 and facilitate the engagement of a penetrating element carried by the delivery catheter 10 with the target penetration site 12 on the IPS wall 114 can ensure successful access of the delivery catheter 10 to the CP angle cistern 138. Conversely, in an IPS 102 with insufficient vessel curvature, the delivery catheter 10 will not travel off-axis from the vessel path to access the CP angle cistern 138, and instead, will skive along the IPS wall 114 without accessing the CP angle cistern 138. Alternatively, the vessel curvature may be so severe that the delivery catheter cannot navigate through the anatomy to access the target penetration site 12 into the CP angle cistern 138. Additional details regarding the importance of vessel curvature to facilitate an endovascular CSF shunt deployment procedure, and related methods and apparatus for such endovascular CSF shunt deployment procedure, are disclosed in WO2017/075544, which is expressly incorporated herein by reference.

As shown in FIGS. 1 and 4, most patients have two IPSs 102 (102L and 102R) and two JVs 106 (106L and 106R). In a very small percentage of patients (e.g., less than 1%), there is no connection between one IPS 102 and the corresponding JV 106. It is highly unlikely, however, that any given patient will lack connections to the corresponding JV 106 on both left and right IPSs 102L, 102R. Assessing the diameter of the IPS 102 from its connection point with the JV 106 to a location distal to the target penetration site 12 on IPS wall 114 is another important anatomic feature to evaluate when planning an endovascular CSF shunt deployment procedure. Vessel diameter of the IPS 102 sufficient to accommodate the largest outer diameter of the endovascular delivery componentry needed for the procedure (e.g., outer diameter of the delivery catheter 10 used during the intended procedure) can ensure procedural success, while insufficient vessel diameter of the IPS 102 can physically obstruct the delivery catheter 10 from advancing through IPS 102 to the target penetration site 12 on the IPS wall 114 and/or accessing CP angle cistern 138.

The foregoing anatomic screening criteria can be used to determine whether a patient has suitable anatomy for the endovascular CSF shunt delivery procedure (e.g., whether the relevant anatomy can physically accommodate the endovascular tools and componentry required to successfully complete the procedure and whether the intended procedure steps and device(s) will avoid injury to critical anatomic structures (e.g., portions of the cardiovascular or central nervous system)). An example of anatomic screening criteria used to assess patent anatomy for an endovascular CSF shunt deployment procedure can include one or more of the following requirements:
1. Depth of the CP angle cistern 138 (i.e., unobstructed CSF space within the ISAS 116) greater than a predetermined distance (e.g., 3 mm, 4 mm, 5 mm) from the target penetration site 12 on IPS wall 114 to the brainstem (e.g., as measured perpendicularly from IPS wall 114 at the target penetration site 12 to the brainstem on an axial image);
2. Distance between the target penetration site 12 on the IPS wall 114 and any arterial or nervous structures residing within the CP angle cistern 138 including, but not limited to, AICA, the vertebral artery, basilar artery, and PICA, greater than a predetermined distance (e.g., 3 mm, 4 mm, 5 mm);
3. Absence of petrous bone overhang at the the target penetration site 12 that may obstruct or prevent the delivery catheter 10 from penetrating the IPS wall 114 at the target penetration site 12 to access the CP angle cistern 138;
4. Vessel diameter of the IPS 102 sufficient to accommodate the delivery catheter 10 from the JV 106 to a location in the IPS 102 distal to the target penetration site 12 (e.g., vessel diameter ≥1.5 mm to accommodate a 4.5 French delivery catheter 10); and
5. Sufficient vessel curvature of the IPS 102 to allow proper trajectory of the delivery catheter 12 off-axis from the vessel path of the IPS 102 to access the CP angle cistern 138.

One or more of the anatomic screening criteria listed above can be used in a procedure planning method to decide whether a patient has anatomy suitable for an endovascular CSF shunt deployment procedure. The minimum depth of the ISAS 116 and vessel diameter dimensions of the IPS 102 required for the procedure planning method can be based, in part, on device dimensions used for the procedure and the minimum distance (or minimum distance plus some factor of safety) within the ISAS 116 to safely accommodate the delivery device (e.g., penetrating element of the delivery catheter 10) while minimizing risk of patient injury.

The anatomic screening criteria listed above can be based on exemplary dimensions and angles of a hypothetical delivery catheter 10 and portions thereof relative to a CP angle cistern 138 when performing an endovascular CSF shunt delivery procedure. For example, as illustrated in FIG. 5, in a hypothetical endovascular CSF shunt delivery procedure, the delivery catheter 10 is illustrated as traversing the IPS wall 114 and dura mater 115 at the target penetration site 12 at a penetration angle of 40°, a penetration length of 5.5 mm, a penetration depth of 3.2 mm, a deployed malecot length of 7.5 mm, and a deployed malecot depth of 5.4 mm.

Imaging methods can be used to capture patient anatomical information that will be evaluated during the procedure planning methods and/or while evaluating patient anatomy during the endovascular procedures disclosed herein. These imaging methods can include magnetic resonance imaging (MRI), computed tomography (CT) scanning, angiography with and without cone-beam computed tomographic (cone-beam CT or CBCT) including CBCT angiography or venography (CBCTA or CBCTV), intravascular ultrasound (IVUS), and intravascular optical coherence tomography (IVOCT).

MRI methods relevant to procedure planning for an endovascular CSF shunt deployment procedure can include an internal auditory canal protocol plus gadolinium, including (a) axial thin slice (i.e., ≤1 mm slice thickness) post-gadolinium from the C2 vertebrae to the top of the clivus bone, and (b) CISS (Constructive Interference in Steady State) or FIESTA-C (Fast Imaging Employing Steady-State Acquisition) sequence through the CP angle cistern 138 to obtain relevant information about IPS vessel diameter and curvature, as well as critical structures within the SAS (e.g., arteries, cranial nerves, brainstem) and their proximity to an intended penetration site on IPS wall 114. Additional MRI protocols or sequences that can be used in embodiments of the procedure planning methods disclosed herein include the following: Sagittal (Sag) T1 (5 mm whole brain); Axial (Ax) T2 (5 mm whole brain); Ax DWI (5 mm whole brain); Ax GRE (5 mm whole brain); Ax T2 FLAIR (5 mm whole brain) (Ax T2 FLAIR PROP); High-res Ax T1 (1 mm or less through IAC, C2 vertebral body to top of orbital rim); +C hi-res Ax T1 (1 mm or less through IAC, C2 vertebral body to top of orbital rim); +C hi-res Cor T1 (1 mm or less slices); +C Ax Fiesta-C T2 3D (GE) or CISS 3D (Siemens) (1 mm thickness); +C Ax T1 (5 mm whole brain); and +C SRS sequence compatible with 1 mm spacing (Brainlab, stealth, and the like).

CT scan methods can include thin slice head CT scans without contrast (i.e., ≤1 mm slice thickness) to evaluate for the presence of petrous bone anatomy that could potentially obstruct delivery catheter access to CP angle cistern 138.

In addition, a transfemoral diagnostic catheter venogram injecting contrast bilaterally in the JV 106 and IPS 102 can be obtained with 3D subtracted rotational venography, and preferably using a cone-beam CT protocol. The venogram imaging allows for an assessment of vessel diameter of the IPS 102 and vessel trajectory relative to CP angle cistern 138, and the cone-beam CT protocols enable volumetric reconstruction of the area of interest to show the relationship between the bony anatomy, the vascular anatomy, and endovascular componentry used in minimally invasive procedures to penetrate the IPS wall 114 to access CP angle cistern 138. PCT Publication No. WO2019/173784 describes methods for obtaining a volumetric reconstruction and utilizing a 3D roadmapping technique to facilitate intra-procedure navigation of endovascular delivery within the vasculature and through an IPS wall 114 at the target penetration location. This provides the operator with important information and guidance on the presence of anatomic landmarks relative to such target location and other local structures of interest including bones, nerves, critical organs (e.g., brain tissue), and venous and arterial structures. Alternatively, or in addition to the diagnostic catheter venogram and volumetric reconstruction detailed above, catheter-based intravascular ultrasound and/or intravascular optical coherence tomography can be used to evaluate vessel diameter of the IPS 102 and vessel trajectory relative to CP angle cistern 138, as well as the relationship between the bony anatomy, the vascular anatomy, and endovascular componentry used in minimally invasive procedures to penetrate the IPS wall 114 to access CP angle cistern 138. One or more of the pre-procedure imaging studies described above (e.g., MRI, CT) or intra-procedure imaging techniques (e.g., venogram with or without cone beam-CT protocols, IVUS, IVOCT) can be combined with fluoroscopy imaging obtained during the endovascular CSF shunt or other procedure to further facilitate image guidance for the operator during the endovascular procedure according to one or more of the methods disclosed in PCT Publication No. WO2019/173784.

For example, 3D-rotational angiography or venography (3DRA or 3DRV), and cone-beam computed tomographic angiography or venography (CBCTA or CBCTV) volumetric reconstructions can be overlaid, registered, combined and/or matched to real-time fluoroscopy imaging using a 3D roadmap technique (e.g., using Siemens syngo 3D Roadmap and syngo Toolbox, or Phillips Dynamic 3D Roadmap) that facilitates an overlay, registration, combination, and/or matching of a point(s) of interest from the 3D or volumetric reconstruction (e.g., DynaCT from Siemens Healthcare, XperCT from Phillips) with real-time 2D fluoroscopy images. Magnetic resonance imaging (MRI) or any of the other imaging modalities described herein provide additional valuable information about the anatomy surrounding intended or target access locations to the subarachnoid space, which MRI or other imaging modality can also be overlaid, registered, combined and/or matched with real-time fluoroscopy and/or a 3D reconstruction.

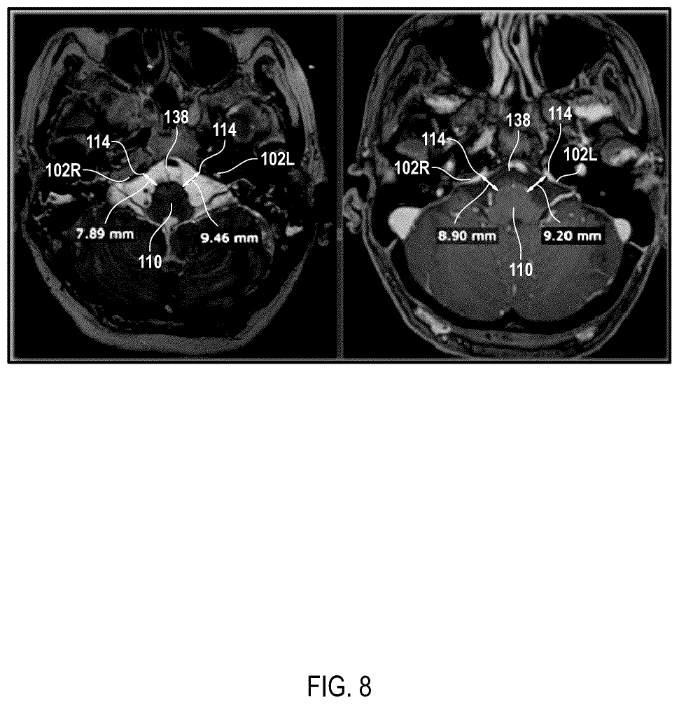

Referring to FIG. 6, one method 200 for procedure planning to evaluate patient anatomy relevant to an endovascular CSF shunt deployment procedure will now be described. It should be appreciated that the sequence of procedure planning steps shown in FIG. 6 are intended only as one non-limiting example, and the steps can be performed in any order prior to the shunt deployment procedure.

The method 200 comprises acquiring patient images relevant to the anatomic region of interest for the endovascular CSF shunt deployment procedure (e.g., IPS 102 and ISAS 116, including CP angle cistern 138) (step 202). This image acquisition step can include obtaining MRI, CT and venogram (with or without CBCT) imaging of the anatomy of interest. All imaging studies can be acquired on the same or different days, and one or both the MRI and CT and be acquired and evaluated in the following steps described below before acquiring the venogram imaging. Alternatively, the venogram can be performed on the day of the intended endovascular procedure following acquisition and evaluation of the MRI and CT imaging, for example, in the same or in a different patient intervention as the endovascular CSF shunt deployment procedure.

The method 200 further comprises reviewing the MRI (e.g., 3D MRI reconstruction) to evaluate the presence or absence of critical structures within the ISAS 116 near the target penetration site of IPS wall 114 (step 204). This review evaluates whether any critical structures (e.g., brainstem, cranial nerves, AICA, vertebral artery, basilar artery, PICA, and other arterial branches) are located in close proximity (e.g., <1 mm, <2 mm, <3 mm, <4 mm, <5 mm, etc.) to an intended trajectory of a delivery catheter penetrating element (e.g., as shown in FIG. 5) passing through IPS wall 114 into the ISAS 116 that could injure such structures and potentially harm the patient. If any critical structures are not a minimum distance from the IPS wall 114 at the target penetration site (step 206), the method 200 will not approve this aspect of the patient anatomy for the endovascular CSF shunt deployment procedure (step 224). In addition, the target penetration site can be adjusted relevant to an anatomic landmark (e.g., bony anatomy proximate IPS wall 114), so that critical structures are avoided during the endovascular CSF shunt deployment procedure. If all of the critical structures are a minimum distance from the IPS wall 114 at the target penetration site (step 206), the method 200 will approve this aspect of the patient anatomy for the endovascular CSF shunt deployment procedure (step 226).

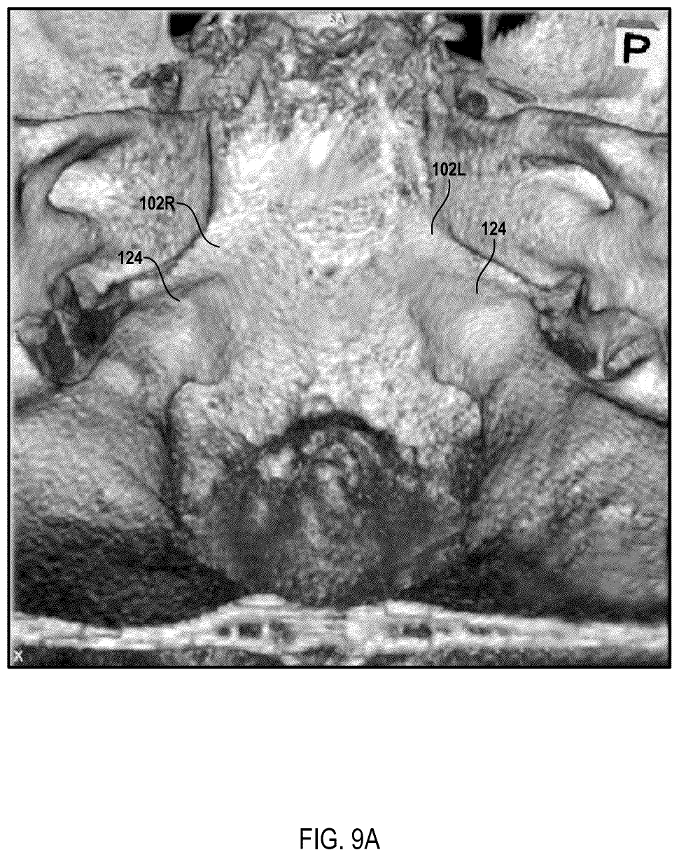

For example, as illustrated in FIG. 7, a measurement can be made on the MRI to evaluate the distance from the IPS wall 114 through CP angle cistern 138 to a vertebral artery 120. As shown, the vertebral artery 120 lies in close proximity (i.e., 1.26 mm) to the IPS wall 114. A delivery catheter penetrating element passing through IPS wall 114 to access CP angle cistern 138 at this location could puncture the vertebral artery 120 potentially resulting in bleeding into the ISAS 116. Thus, such anatomy would not be suitable for an endovascular CSF deployment due to an increased potential for patient injury. Conversely, if the vertebral artery 120 were at least some minimum distance away from a target penetration site on IPS wall 114 (e.g., ≥5 mm) and/or outside the intended trajectory of the delivery catheter through IPS wall 114 into CP angle cistern 138, such anatomy would be suitable for an endovascular CSF deployment due to the minimized potential for patient injury.

The method 200 further comprises reviewing the MRI (e.g., MRI reconstruction (MPR)) to evaluate whether the left and right IPS vessel diameters have sufficient diameter to accommodate the endovascular delivery componenty (and in this case, the delivery catheter 10) required to complete the endovascular CSF shunt deployment procedure (step 208). The IPS vessel diameter must be large enough to allow navigation of the device having the largest outer diameter required for the endovascular CSF shunt deployment procedure (in this case, the delivery catheter 10) through IPS 102 to the target penetration site on IPS wall 114. For example, if a 4.5 French delivery catheter will be used, IPS diameters should be greater than 1.5 mm in diameter from its connection with the jugular vein to a location distal to the target penetration site to access CP angle cistern 138. If the IPS vessel diameter is not large enough to allow navigation of the delivery catheter (step 210), the method 200 will not approve this aspect of the patient anatomy for the endovascular CSF shunt deployment procedure (step 224). If the IPS vessel diameter is large enough to allow navigation of the delivery catheter (step 210), the method 200 will approve this aspect of the patient anatomy for the endovascular CSF shunt deployment procedure (step 226).

The method 200 further comprises reviewing the MRI (e.g., MPR)) to evaluate the depth of CP angle cistern 138 proximate a target penetration site on the IPS wall 114 (step 212). The requirement for this depth measurement can be based, in part, on device dimensions used for the endovascular CSF shunt deployment procedure and the minimum distance (or minimum distance plus some factor of safety) within the ISAS 116 to safely accommodate the delivery device (e.g., delivery catheter penetrating element or the portion of the CSF shunt extending into the CP angle cistern 138) while minimizing risk of patient injury. Example device dimensions including distances of device portions with a hypothetical CP angle cistern 138 relevant to step 212 of the method 200 are shown in FIG. 5. If the depth of the CP angle cistern 138 proximate the target penetration site on the IPS wall 114 (measured by the orthogonal distance between the border of the IPS wall 114 and the border of the brain stem) is less than a minimum distance (step 214), the method 200 will not approve this aspect of the patient anatomy for the endovascular CSF shunt deployment procedure (step 224). If the depth of the CP angle cistern 138 within the ISAS 116 proximate the target penetration site on the IPS wall 114 is at least the minimum distance (step 214), the method 200 will approve this aspect of the patient anatomy for the endovascular CSF shunt deployment procedure (step 226).

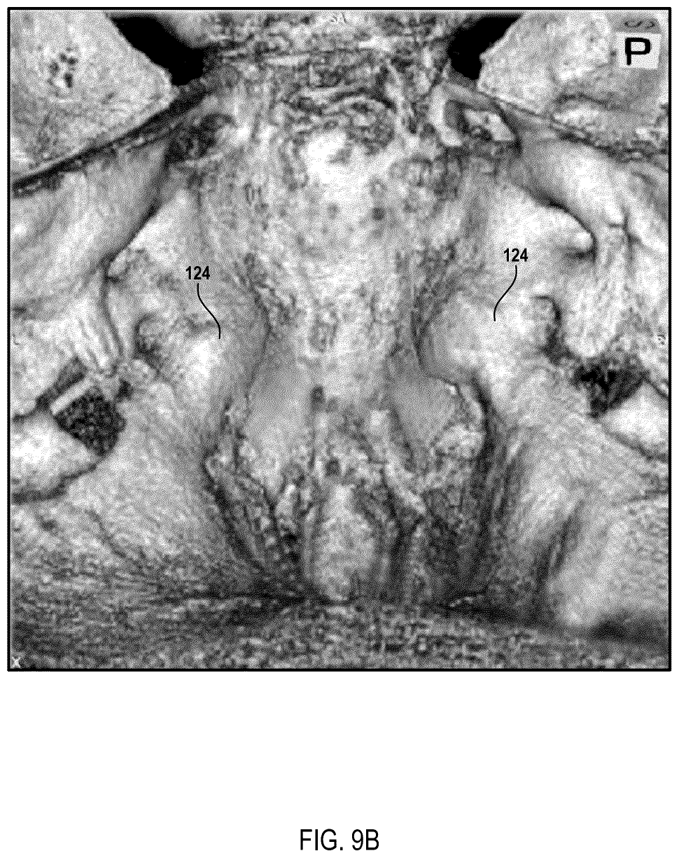

For example, as illustrated in FIG. 8, all measurements show the depth of the CP angle cistern 138 from the IPS wall 114 of the left and right IPSs 102L, 102R to the brain brainstem 112 greater than 5 mm, which is sufficient to safely accommodate an endovascular CSF shunt deployment procedure without risk of brainstem, arterial or nerve injury. Conversely, patient anatomy exhibiting a smaller depth of the CP angle 138 (e.g., 1 mm between IPS wall 114 and brainstem 112) would not safely accommodate the penetrating element of the delivery catheter or the portion of the CSF shunt device extending into the CP angle cistern 138, and the method 200 would not approve such patient anatomy for the endovascular CSF shunt deployment procedure.

The method 200 further comprises reviewing the CT imaging (e.g., cone-beam CT providing a 3D reconstruction of the relevant anatomy) to evaluate for the presence or absence of petrous or other bony anatomy that could prevent penetration of the delivery catheter through IPS wall 114 into CP angle cistern 138 (step 216). If there is a presence of petrous or other bony anatomy that could prevent penetration of the delivery catheter through the IPS wall 114 into the CP angle cistern 138 (step 218), the method 200 will not approve this aspect of the patient anatomy for the endovascular CSF shunt deployment procedure (step 224). If there is an absence of petrous or other bony anatomy that could prevent penetration of the delivery catheter through the IPS wall 114 into the CP angle cistern 138 (step 218), the method 200 will approve this aspect of the patient anatomy for the endovascular CSF shunt deployment procedure (step 226).

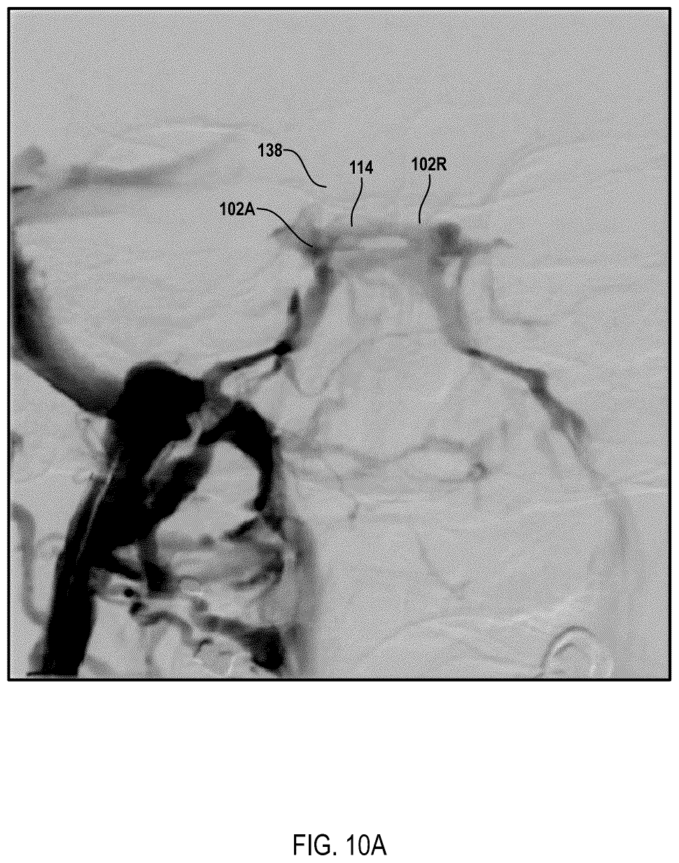

For example, as illustrated in FIG. 9A, the CT scan of patient anatomy evaluated in step 216 reveals the absence of any petrous or other bony structure 124 that could prevent delivery catheter access through IPS wall 114 into CP angle cistern 138 from both the left IPS 102L and the right IPS 102R. In this case, the method 200 can deem both the left IPS 102L and right IPS 102R suitable for the endovascular CSF shunt deployment procedure. In contrast, as illustrated in FIG. 9B, the CT scan of patient anatomy evaluated in step 216 reveals that both the left IPS 102L and the right IPS 102R have petrous bone 124 overhanging the portion of the IPS vessel path or IPS wall 114 where a delivery catheter would attempt to access CP angle cistern 138. Thus, the method 200 would deem both the left IPS 102L and the right IPS 102R unsuitable for the endovascular CSF shunt deployment procedure, because the bony overhang would prevent the delivery catheter from passing through IPS wall 114 into CP angle cistern 138.

The method 200 further comprises reviewing venogram imaging to confirm the vessel diameter of the IPS 102 and assess the extent of the first curved portion 102A of the IPS 102 relative to the target penetration site on the IPS wall 114 (step 220). If the venogram imaging reveal that the first curved portion 102 of the IPS 102 would not facilitate off-axis tracking of the delivery catheter from the IPS path to successfully penetrate through the IPS wall 114a to the CP angle cistern 138 (step 222), the method 200 will not approve this aspect of the patient anatomy for the endovascular CSF shunt deployment procedure (step 224). If the venogram imaging reveal that the first curved portion 102A of the IPS 102 would facilitate off-axis tracking of the delivery catheter from the IPS path to successfully penetrate through the IPS wall 114a to the CP angle cistern 138 (step 222), the method 200 will approve this aspect of the patient anatomy for the endovascular CSF shunt deployment procedure (step 226). Alternatively, MRI, any of the cone-beam CT protocols with or without 3D reconstruction, IVUS, and/or IVOCT can be used to confirm the vessel diameter of the IPS 102 in steps 208-210 and assess the extent of a first curved portion 102A of the IPS 102 relative to the target penetration site on the IPS wall 114 in steps 220-222.

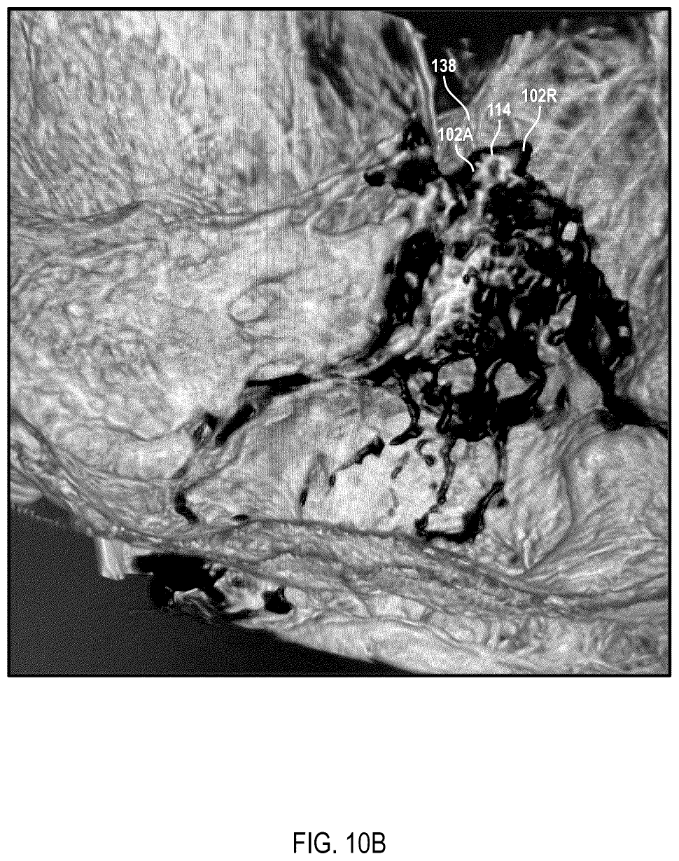

For example, referring to FIGS. 10A-10B, an x-ray image and cone-beam CT image of the target penetration site on the IPS wall 114 acquired during the venogram shows an optimal curvature of the right IPS 102R relative to the CP angle cistern 138, which would facilitate off-axis tracking of the delivery catheter from the IPS path at the first curved portion 102A to successfully access angle cistern 138. The method 200 would approve this specific patient anatomy for an endovascular CSF shunt deployment procedure.

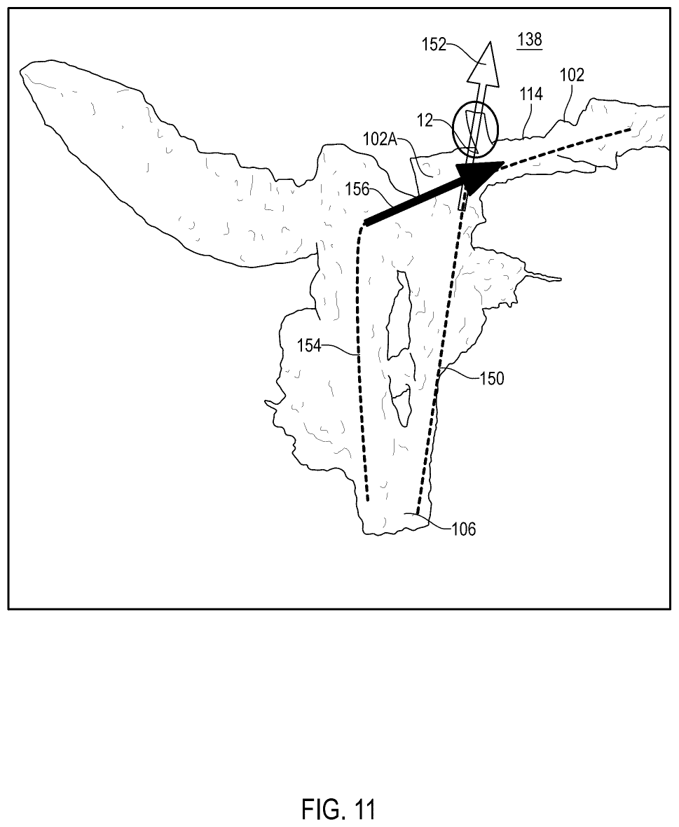

Referring to FIG. 11, a cone-beam CT image of a target penetration site on the IPS wall 114 shows two pathways 150, 154 (shown by dashed lines) from JV 106 into the IPS that potentially provide access to the CP angle cistern 138. The pathway 150 from the JV 106 ending with the solid arrow 152 shows an optimal curvature of the IPS 102 relative to the CP angle cistern 138, which would facilitate off-axis tracking of the delivery catheter along the IPS pathway 150 at the first curved portion 102A to successfully access the CP angle cistern 138 via the target penetration site 12 in the IPS wall 114. The method 200 would approve this aspect of patient anatomy for an endovascular CSF shunt deployment procedure. In contrast, the pathway 154 from the JV 106 ending with the solid arrow 156 shows an inadequate curvature of the IPS 102 relative to the CP angle cistern 138, which would not facilitate off-axis tracking of the delivery catheter along the IPS pathway 154 at the first curved portion 102A to successfully access the CP angle cistern 138 via the target penetration site 12 in the IPS wall 114. Instead, the penetrating element of a delivery catheter advanced along the IPS pathway 154 would skive the IPS wall 114 along the IPS pathway 154 rather than penetrate through IPS wall 114 to access CP angle cistern 138.

Although particular embodiments have been shown and described herein, it will be understood by those skilled in the art that they are not intended to limit the disclosed inventions, and it will be appreciated by those skilled in the art that various changes, permutations, and modifications may be made (e.g., the dimensions of various parts, combinations of parts) without departing from the scope of thereof, which is to be defined only by the following claims and their equivalents. The specification and drawings are, accordingly, to be regarded in an illustrative rather than restrictive sense.

1. A method for planning an endovascular procedure to access an intracranial subarachnoid space (ISAS) through a blood vessel wall of a patient, the method comprising:
obtaining magnetic resonance (MR) imaging of the blood vessel and the ISAS;
measuring in the MRI imaging an unobstructed depth of the ISAS at a target penetration site or a distance from a target penetration site of the blood vessel to one or more critical structures within the ISAS; and
approving or disapproving the patient for the endovascular procedure based on the measured unobstructed depth of the ISAS at the target penetration site or the measured distance from the target penetration site of the blood vessel to the one or more critical structures within the ISAS.

2. The method of claim 1, wherein the one or more critical structures comprise an artery, a nerve, or a brainstem of the patient.

3. The method of claim 1, further comprising:
measuring in the MRI imaging a diameter of the blood vessel the target penetration site; and
approving or disapproving the patient for the endovascular procedure based on measured diameter of the blood vessel.

4. The method of claim 3, further comprising:
comparing the measured blood vessel diameter to a largest outer diameter of a delivery catheter intended to be used in the endovascular procedure; and
approving or disapproving the patient for the endovascular procedure based on the comparison.

5. The method of claim 1, further comprising:
obtaining computed tomography (CT) imaging of the blood vessel and the ISAS;
assessing the CT imaging for the presence or absence of bony anatomy near the target penetration site of the blood vessel; and
approving or disapproving the patient for the endovascular procedure based on assessment.

6. The method of claim 1, further comprising:
obtaining a venogram of the blood vessel including the target penetration site;
evaluating the venogram for a curvature of the blood vessel proximate the target penetration site; and
approving or disapproving the patient for the endovascular procedure based on the evaluation.

7. The method of claim 6, wherein obtaining the venogram comprises acquiring a cone-beam computed tomography (CT) imaging of the blood vessel and ISAS, the method further comprising evaluating the cone-beam CT imaging for a pathway of the blood vessel to determine whether a delivery catheter, when advanced through the pathway, will travel off-axis from the pathway at the target penetration site to access the ISAS, and approving the patient for the endovascular procedure based on whether the delivery catheter will travel off-axis from the pathway at the target penetration site to access the ISAS.

8. The method of claim 1, wherein the blood vessel is an intracranial venous sinus.

9. The method of claim 8, wherein the intracranial venous sinus is an inferior petrosal sinus (IPS).

10. The method of claim 1, wherein the ISAS comprises a cerebellopontine (CP) angle cistern.

11. The method of claim 1, further comprising:
approving the patient for the endovascular procedure; and
performing the endovascular procedure on the patient.

12. The method of claim 11, wherein the endovascular procedure comprises deploying an endovascular cerebrospinal fluid (CSF) shunt within the ISAS.

13. The method of claim 11, wherein the endovascular procedure comprises administering a therapeutic agent into the ISAS.

14. A method for planning an endovascular procedure to access an intracranial subarachnoid space (ISAS) through a blood vessel wall of a patient, the method comprising:
obtaining imaging of the blood vessel and the ISAS;
performing at least two of the following diagnostic steps to obtain anatomic screening criteria:
measuring in the imaging an unobstructed depth of the ISAS at the target penetration site;
measuring in the imaging a distance from a target penetration site of the blood vessel to one or more critical structures within the ISAS;
measuring in the imaging a diameter of the blood vessel the target penetration site;
assessing the imaging for the presence or absence of bony anatomy near the target penetration site of the blood vessel; and
evaluating the imaging for a curvature of the blood vessel proximate the target penetration site; and
approving or disapproving the patient for the endovascular procedure based on the anatomic screening criteria.

15. The method of claim 14, wherein at least three of the diagnostic steps are performed to obtain the anatomic screening criteria.

16. The method of claim 14, wherein the blood vessel is an intracranial venous sinus.

17. The method of claim 16, wherein the intracranial venous sinus is an inferior petrosal sinus (IPS).

18. The method of claim 14, wherein the ISAS comprises a cerebellopontine (CP) angle cistern.

19. The method of claim 14, further comprising:
approving the patient for the endovascular procedure; and
performing the endovascular procedure on the patient.

20. The method of claim 19, wherein the endovascular procedure comprises an endovascular cerebrospinal fluid (CSF) shunt deployment procedure.

21. The method of claim 19, wherein the endovascular procedure comprises administering a therapeutic agent into the ISAS.
